(19) United States
(12) Patent Application Publication
(10) Pub. No.: US 2026/0335644 A1
(43) Pub. Date: Oct. 29, 2026

(54) COCHLEAR IMPLANT SYSTEM AND METHOD OF CONTROLLING THE SAME

(71) Applicant: Jeong-Pyo Han, Seoul (KR)

(72) Inventor: Jeong-Pyo Han, Seoul (KR)

(21) Appl. No.: 19/700,189

(22) Filed: Jun. 6, 2026

(30) Foreign Application Priority Data
Apr. 10, 2025 (KR) 10-2025-0047012

(51) Int. Cl. A61N 1/36; A61N 1/05

(52) U.S. Cl. CPC A61N 1/36039; A61N 1/0541

(57) ABSTRACT

Provided are a cochlear implant system and a method of controlling the same. The cochlear implant system includes an internal device configured to be implanted in an outer ear of a user and including a first coil, and a sound processor configured to modulate a sound signal according to hearing characteristics of the user and transmit the modulated sound signal to the internal device in a non-contact manner. The sound processor may detect the hearing characteristics of the user using a first test signal and determine a modulation algorithm for the sound signal according to the detected hearing characteristics. The sound processor may also detect state information of the internal device using a second test signal and generate a notification signal according to the detected state information.

### TECHNICAL FIELD

The present disclosure relates generally to a cochlear implant system and a method of controlling the same, and more particularly, to a cochlear implant system capable of improving a user's sound recognition ability by processing a sound signal according to hearing characteristics of the user, and capable of detecting a state of an internal device implanted in a cochlea or an ear region of the user.

### BACKGROUND

A human ear, which enables a person to recognize sound, generally includes an outer ear, a middle ear, and an inner ear. External sound is transmitted from the outer ear to an eardrum of the middle ear through vibration of air, and is then transmitted to an oval window of a cochlea through vibration of auditory ossicles.

An organ of Corti generates an electrical signal through vibration of fluid in the cochlea. Movement of a tectorial membrane generates a receptor potential in hair cells, and the receptor potential is transmitted to a spiral ganglion through dendrites.

A signal from the spiral ganglion stimulates neurons of an auditory nerve and is ultimately transmitted to the brain, thereby enabling recognition of external sound.

When hair cells in the cochlea are damaged or otherwise abnormal, hearing ability may be reduced, making it difficult for a user to recognize external sound.

To address such hearing loss, cochlear implant technologies have been developed in which an artificial cochlear device is implanted in a body to externally provide electrical signals that would otherwise be generated by hair cells.

A cochlear implant may generally include an internal device implanted in the user's body and a sound processor configured to transmit external sound to the internal device.

The internal device may include an electrode configured to transmit an electrical signal to the cochlea in a non-contact or wireless manner with respect to the sound processor. However, pressure ulcers or other tissue problems may occur at or near an implantation site.

In addition, a conventional cochlear implant may operate within a frequency band lower or narrower than a normal hearing frequency range. Further, because a perceivable frequency range may vary from user to user, it may be difficult for a conventional cochlear implant system to process sound in a manner that appropriately reflects individual hearing characteristics of each user.

### Problems to be Solved

An objective of the present disclosure is to provide a cochlear implant system and a method of controlling the same that are capable of improving a user's sound recognition ability by reflecting hearing characteristics of the user.

Another objective of the present disclosure is to provide a cochlear implant system and a method of controlling the same that are capable of detecting a state of an internal device implanted in a body.

### SUMMARY

In order to achieve the above-described objectives, the present disclosure may provide a cochlear implant system including: an internal device configured to be implanted in an outer ear of a user and including a first coil; and a sound processor configured to modulate a sound signal according to hearing characteristics of the user and transmit the modulated sound signal to the internal device in a non-contact manner.

The sound processor may include: a second coil configured to exchange electrical signals with the first coil through mutual induction; a microphone configured to receive the sound signal; a sound modulator configured to divide the sound signal into a plurality of frequency bands and modulate signals in the plurality of frequency bands according to the hearing characteristics of the user; a transmitter configured to transmit the modulated signals in the plurality of frequency bands to the internal device through the second coil; a characteristic detector configured to detect an induced signal induced in the second coil by a test signal and determine the hearing characteristics of the user or a state of the internal device based on the induced signal; and a controller configured to control the sound modulator, the transmitter, and the characteristic detector.

The test signal may include a first test signal for detecting the hearing characteristics of the user and a second test signal for detecting whether the internal device is defective.

The first test signal may be provided at an initial operation time after the internal device is implanted.

The second test signal may be provided at predetermined periodic intervals.

The sound modulator may be configured to, based on the hearing characteristics of the user detected using the first test signal, divide the sound signal into a user-recognizable signal included in a user-recognizable frequency range and a user-unrecognizable signal included in a user-unrecognizable frequency range outside the user-recognizable frequency range.

The sound modulator may be configured to modulate the user-unrecognizable signal into the user-recognizable frequency range.

The sound modulator may be configured to modulate the user-unrecognizable signal such that the user-unrecognizable signal overlaps the user-recognizable signal.

The sound modulator may be configured to reduce the user-recognizable signal at a predetermined ratio and modulate the user-unrecognizable signal into an available frequency band in which the reduced user-recognizable signal is not present.

In addition, the present disclosure may provide a method of controlling a cochlear implant system including a sound processor configured to communicate with, in a non-contact manner, an internal device implanted in an outer ear of a user, the method including: transmitting a first test signal to the internal device to detect hearing characteristics of the user; determining a modulation algorithm for a sound signal according to the hearing characteristics of the user; receiving the sound signal through a microphone; modulating the sound signal according to the modulation algorithm; and transmitting the modulated sound signal to the internal device.

The method of controlling the cochlear implant system may further include transmitting a second test signal to the internal device to detect state information of the internal device, and generating a notification signal according to the state information of the internal device.

The modulating of the sound signal may include, based on the hearing characteristics of the user detected using the first test signal, dividing the sound signal into a user-recognizable signal included in a user-recognizable frequency range and a user-unrecognizable signal included in a user-unrecognizable frequency range outside the user-recognizable frequency range.

The modulating of the sound signal may include modulating the user-unrecognizable signal into the user-recognizable frequency range.

The modulating of the sound signal may include modulating the user-unrecognizable signal such that the user-unrecognizable signal overlaps the user-recognizable signal.

The modulating of the sound signal may include reducing the user-recognizable signal at a predetermined ratio, and modulating the user-unrecognizable signal into an available frequency band in which the reduced user-recognizable signal is not present.

### Advantageous Effects

According to embodiments of the present disclosure, a user's sound recognition ability may be improved by reflecting the user's hearing characteristics in processing a sound signal.

In addition, according to embodiments of the present disclosure, a state of an internal device implanted in the body may be detected and controlled.

### BRIEF DESCRIPTION OF THE DRAWINGS

FIG. 1 is a schematic configuration diagram illustrating a cochlear implant system according to an embodiment of the present disclosure.

FIG. 2 is a view illustrating an internal device in the cochlear implant system according to an embodiment of the present disclosure.

FIG. 3 is a block diagram illustrating a sound processor in the cochlear implant system according to an embodiment of the present disclosure.

FIG. 4 is a flowchart illustrating an example of a method of controlling the cochlear implant system according to an embodiment of the present disclosure.

FIG. 5 is a signal waveform diagram illustrating an example of a process of determining hearing characteristics of a user in the method of controlling the cochlear implant system according to an embodiment of the present disclosure.

FIGS. 6 and 7 are diagrams illustrating an example of a method of modulating a sound signal in the method of controlling the cochlear implant system according to an embodiment of the present disclosure.

FIG. 8 is a diagram illustrating another example of a method of modulating a sound signal in the method of controlling the cochlear implant system according to an embodiment of the present disclosure.

### DETAILED DESCRIPTION

Hereinafter, some embodiments of the present disclosure will be described in detail with reference to exemplary drawings.

In assigning reference numerals to components in the drawings, the same components may be designated by the same reference numerals as much as possible even when they are illustrated in different drawings. In describing the present disclosure, when a detailed description of a related known configuration or function is determined to obscure the gist of the present disclosure, the detailed description thereof may be omitted. When the terms “include,” “have,” “comprise,” or the like are used in the present specification, other components may be added unless the term “only” is used. When a component is expressed in the singular, the singular expression may include the plural unless expressly stated otherwise.

In describing components of the present disclosure, terms such as first, second, A, B, (a), (b), and the like may be used. These terms are used merely to distinguish one component from another component, and the nature, order, sequence, number, or the like of the corresponding component is not limited by such terms.

In describing a positional relationship between components, when two or more components are described as being “connected,” “coupled,” or “joined,” it should be understood that the two or more components may be directly “connected,” “coupled,” or “joined,” but another component may also be further “interposed” between the two or more components so that the two or more components are “connected,” “coupled,” or “joined.” Here, the other component may be included in at least one of the two or more components that are “connected,” “coupled,” or “joined” to each other.

In describing a temporal flow relationship related to components, an operation method, a manufacturing method, or the like, for example, when a temporal order or flow order is described using terms such as “after,” “subsequently,” “next,” “before,” or the like, non-continuous cases may also be included unless the terms “immediately” or “directly” are used.

Meanwhile, when a numerical value or corresponding information thereof, such as a level, is mentioned with respect to a component, the numerical value or the corresponding information may be interpreted as including an error range that may occur due to various factors, such as process factors, internal or external impacts, noise, or the like, even when there is no separate express description.

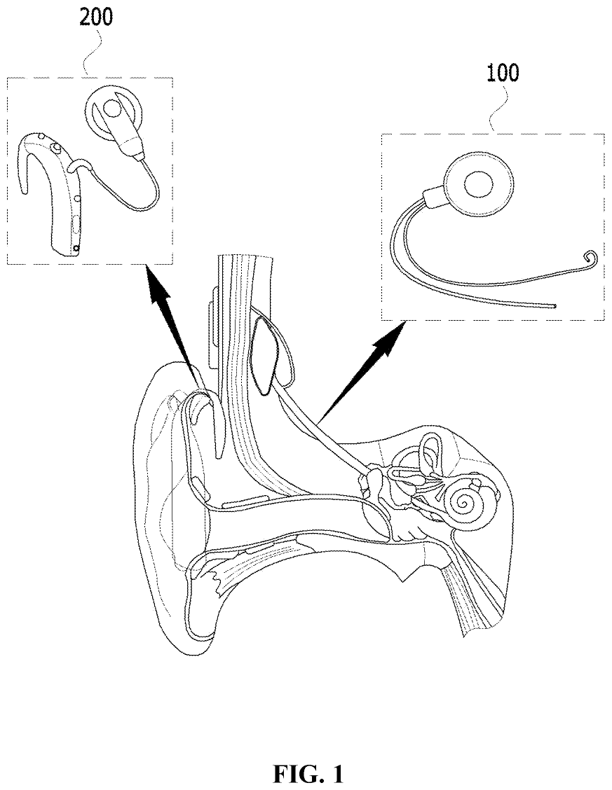

FIG. 1 is a schematic configuration diagram of a cochlear implant system according to an embodiment of the present disclosure.

Referring to FIG. 1, the cochlear implant system according to an embodiment of the present disclosure may include an internal device 100 implanted in an outer ear of a user, and a sound processor 200 configured to transmit and receive signals to and from the internal device 100 in a non-contact manner.

The sound processor 200 and the internal device 100 are coupled to each other in a non-contact manner by coils, and may wirelessly transmit and receive signals to and from each other.

The internal device 100 delivers electrical stimulation directly to a cochlea according to an electrical signal transmitted from the sound processor 200.

The sound processor 200 may include a sound modulator configured to modulate an external sound signal, a transmitter configured to transmit a signal modulated by the sound modulator to the internal device 100, a characteristic detector configured to detect a signal generated by the internal device 100 and determine a state of the internal device 100 or hearing characteristics of the user, and a controller.

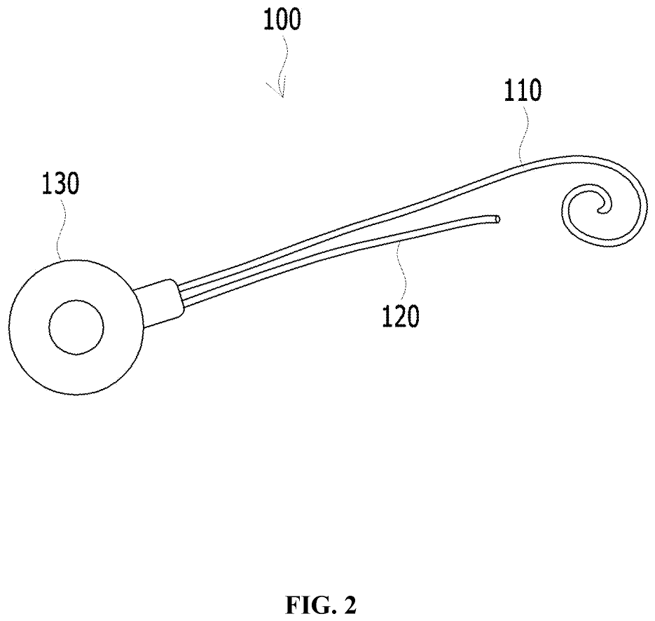

FIG. 2 is a view illustrating the internal device in the cochlear implant system according to an embodiment of the present disclosure.

Referring to FIG. 2, in the cochlear implant system according to an embodiment of the present disclosure, the internal device 100 may include an active electrode 110, a reference electrode 120, and a first coil 130.

The active electrode 110 may extend from the first coil 130 through a minute gap between skin of the outer ear and an ear bone, through an eardrum of a middle ear, and to the cochlea. In this case, an end of the active electrode 110 may have a spiral shape and may be connected to contact a spiral ganglion of the cochlea.

In addition, the reference electrode 120 may extend from the first coil 130 and may be inserted into skin of the outer ear.

The first coil 130 may convert an electrical signal transmitted from the sound processor 200, and the converted electrical signal may be delivered to the spiral ganglion through the active electrode 110.

The internal device 100 of the present disclosure may receive an electrical signal transmitted from the sound processor 200 through the first coil 130, thereby delivering external sound so that the user may recognize the external sound. In addition, the internal device 100 of the present disclosure may transmit, back to the sound processor 200, an induced signal generated by the electrical signal of the sound processor 200 and the electrodes 110 and 120, thereby allowing the sound processor 200 to detect a characteristic value of the internal device 100.

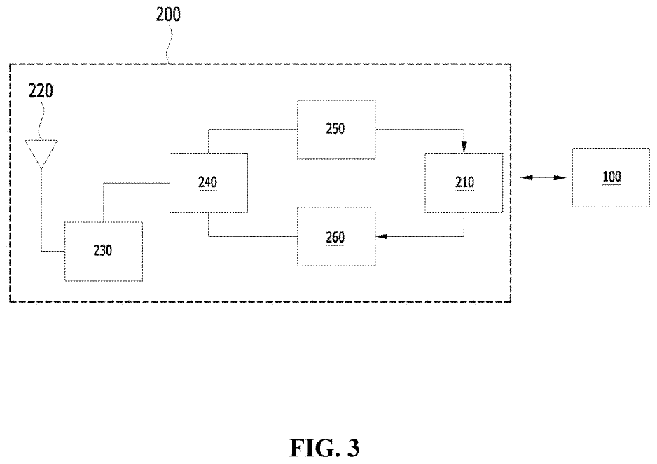

FIG. 3 is a block diagram illustrating the sound processor in the cochlear implant system according to an embodiment of the present disclosure.

Referring to FIG. 3, in the cochlear implant system according to an embodiment of the present disclosure, the sound processor 200 may include a second coil 210, a microphone 220, a sound modulator 230, a controller 240, a transmitter 250, and a characteristic detector 260.

The second coil 210 may exchange electrical signals with the first coil 130 of the internal device 100 through mutual induction in a non-contact manner.

The microphone 220 receives a sound signal generated externally. In this case, the microphone 220 may be integrally formed inside the sound processor 200 or may be formed as a separate type so as to be connectable to the sound processor 200.

The sound modulator 230 may divide the sound signal received through the microphone 220 into a plurality of frequency bands, and may modulate a signal of each frequency band according to hearing characteristics of the user.

The controller 240 may transmit the signal modulated by the sound modulator 230 to the internal device 100 through the transmitter 250 and the second coil 210. In addition, the controller 240 may control the transmitter 250 to generate a test signal for detecting the hearing characteristics of the user or a state of the internal device 100.

The characteristic detector 260 may transmit the test signal to the first coil 130 of the internal device 100, detect an induced signal induced in the second coil 210 by the test signal, and determine the hearing characteristics of the user or the state of the internal device 100.

For example, the controller 240 may detect the hearing characteristics of the user through the characteristic detector 260 using a first test signal, and may control a modulation algorithm of the sound modulator 230 to modulate the sound signal according to the hearing characteristics of the user.

In addition, the controller 240 may detect state information of the internal device 100 through the characteristic detector 260 using a second test signal, and may generate a notification signal for replacement or inspection of the internal device 100 according to the state information of the internal device 100.

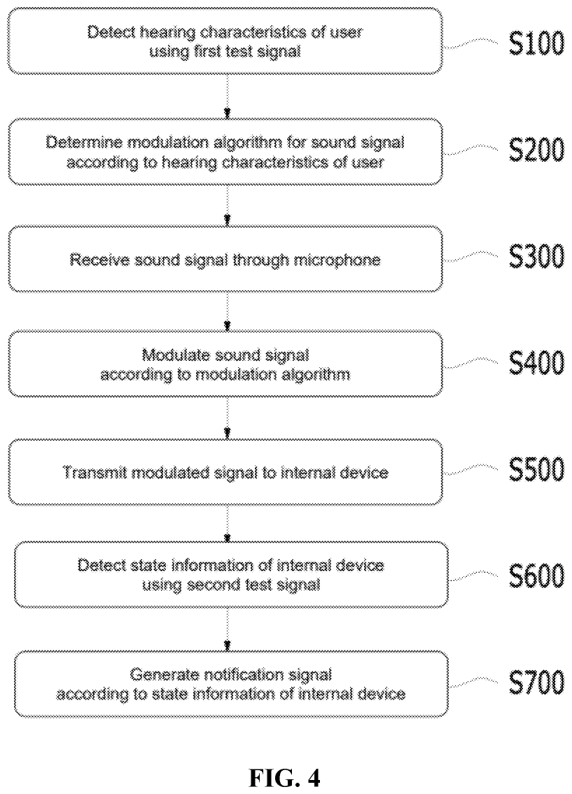

FIG. 4 is a flowchart illustrating an example of a method of controlling the cochlear implant system according to an embodiment of the present disclosure.

Referring to FIG. 4, the method of controlling the cochlear implant system according to an embodiment of the present disclosure may include detecting the hearing characteristics of the user using the first test signal (S100), determining a modulation algorithm of the sound signal according to the hearing characteristics of the user (S200), receiving the sound signal through the microphone 220 (S300), modulating the sound signal according to the modulation algorithm (S400), transmitting the modulated signal to the internal device 100 (S500), detecting the state information of the internal device 100 using the second test signal (S600), and generating the notification signal according to the state information of the internal device 100 (S700).

The detecting of the hearing characteristics of the user using the first test signal (S100) is a process of searching for the hearing characteristics of the user in order to determine an algorithm for modulating an external sound signal after the internal device 100 is implanted inside the outer ear of the user.

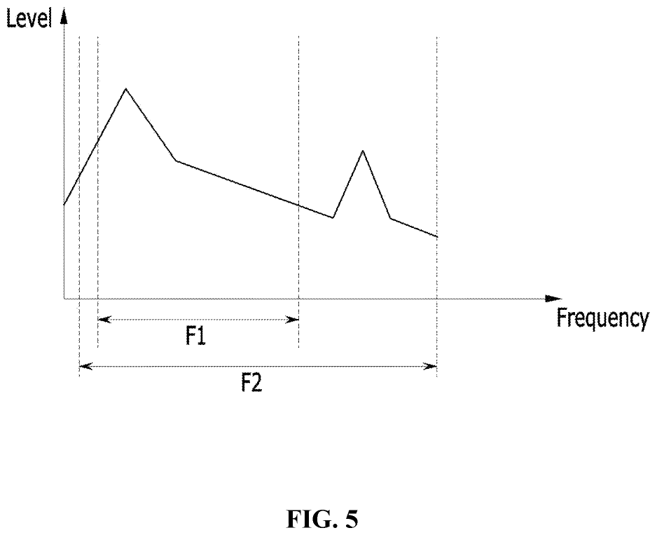

FIG. 5 is a signal waveform diagram illustrating an example of a process of determining the hearing characteristics of the user in the method of controlling the cochlear implant system according to an embodiment of the present disclosure.

Referring to FIG. 5, the detecting of the hearing characteristics of the user using the first test signal (S100) may be performed at an initial operation time of the sound processor 200 in a state in which the internal device 100 is implanted inside the outer ear of the user.

In this case, the sound processor 200 may search for a user-recognizable frequency F1 that the user can recognize within a range of an audible frequency F2 of about 20 Hz to 20,000 Hz. To this end, the sound processor 200 may transmit, to the internal device 100, a first test at a frequency adjacent to 20 Hz and at a frequency adjacent to 20,000 Hz, and may determine a range of the user-recognizable frequency F1 according to whether the user recognizes the first test.

For example, through this process, the user may search for a range of the user-recognizable frequency F1 of 40 Hz to 17,000 Hz. In this case, among the audible frequency F2 of 20 Hz to 20,000 Hz, the range of the user-recognizable frequency F1 may be determined as 40 Hz to 17,000 Hz.

The determining of the modulation algorithm of the sound signal according to the hearing characteristics of the user (S200) is a process of determining an algorithm for modulating the sound signal according to the hearing characteristics of the user.

For example, when the range of the user-recognizable frequency F1 is 40 Hz to 17,000 Hz, the modulation algorithm of the sound signal may be determined so that, among the audible frequency F2 of 20 Hz to 20,000 Hz, sound signals of 20 Hz to 40 Hz and 17,000 Hz to 20,000 Hz that cannot be recognized by the user are modulated into the range of the user-recognizable frequency F1. In this case, a sound signal included in the range of 40 Hz to 17,000 Hz, which is the user-recognizable frequency F1, may be maintained as it is, or may be changed in frequency at a predetermined ratio in consideration of the modulated signal.

The receiving of the sound signal through the microphone 220 (S300) is a process in which the user listens to an external sound signal while wearing the sound processor 200. In this case, the sound signal included in the range of the audible frequency F2 may include a user-recognizable signal included in the range of the user-recognizable frequency F1 and a user-unrecognizable signal outside the range of the user-recognizable frequency F1.

The modulating of the sound signal according to the modulation algorithm (S400) is a process of modulating the external sound signal by applying the modulation algorithm determined according to the hearing characteristics of the user.

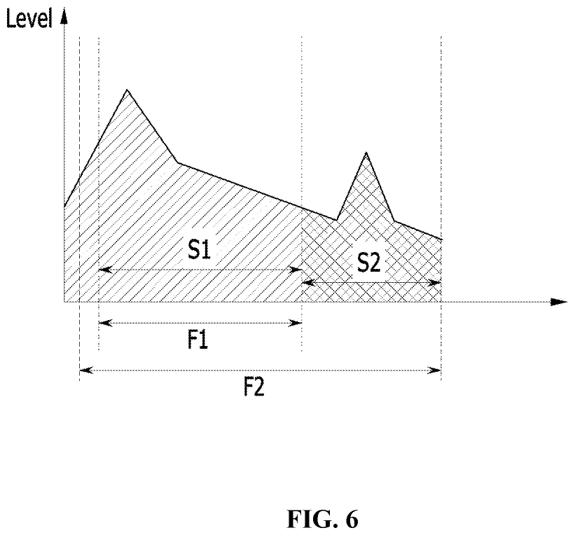

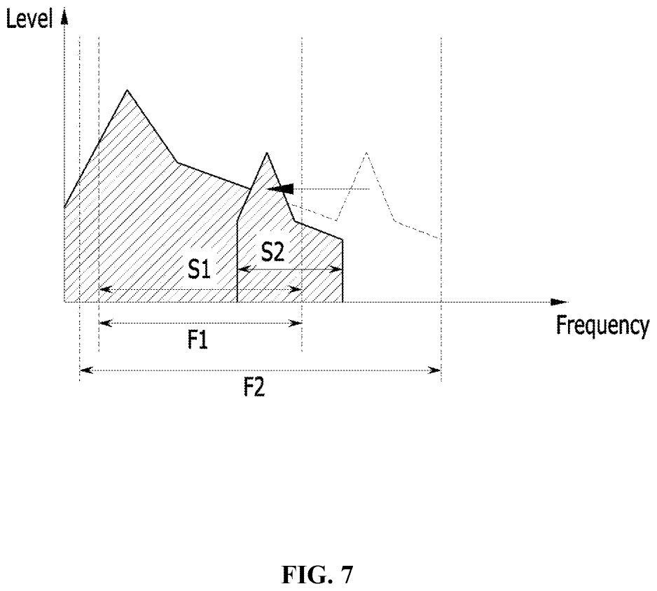

FIGS. 6 and 7 are diagrams illustrating an example of one method of modulating the sound signal in the method of controlling the cochlear implant system according to an embodiment of the present disclosure.

Referring to FIG. 6, the sound processor 230 of the cochlear implant system according to an embodiment of the present disclosure may classify the sound signal received from the microphone 220 into a user-recognizable signal S1 included in the range of the user-recognizable frequency F1 and a user-unrecognizable signal S2 outside the range of the user-recognizable frequency F1.

Here, the user-unrecognizable signal S2 may be regarded as a signal that is outside the range of the user-recognizable frequency F1 but is included in the range of the audible frequency F2.

In this case, when the sound signal is transmitted to the internal device 100, the user may hear the user-recognizable signal S1 included in the range of the user-recognizable frequency F1, but may not hear the user-unrecognizable signal S2 outside the user-recognizable frequency F1.

Accordingly, the cochlear implant system of the present disclosure may control the user-unrecognizable signal S2, which the user cannot recognize among the sound signals of the audible frequency F2, to be recognized by the user through the internal device 100 by modulating the user-unrecognizable signal S2 into the range of the user-recognizable frequency F1.

Referring to FIG. 7, the method of controlling the cochlear implant system according to an embodiment of the present disclosure may modulate the user-unrecognizable signal S2, which the user cannot recognize among the sound signals of the audible frequency F2, into the range of the user-recognizable frequency F1.

For example, in a state in which the user-recognizable signal S1 included in the range of the user-recognizable frequency F1 is maintained as it is, the frequency of the user-unrecognizable signal S2 may be changed into the range of the user-recognizable frequency F1.

In this case, the user-unrecognizable signal S2 may overlap the user-recognizable signal S1 within the range of the user-recognizable frequency F1. In this case, when a high-level sound signal is redundantly present in a region in which the user-unrecognizable signal S2 and the user-recognizable signal S1 overlap, the sound signal may be recognized by the user as noise.

To solve this problem, another method of controlling the cochlear implant system of the present disclosure may change the frequency of the user-recognizable signal S1 so as not to overlap the user-unrecognizable signal S2 in the process of changing the frequency of the user-unrecognizable signal S2 into the range of the user-recognizable frequency F1.

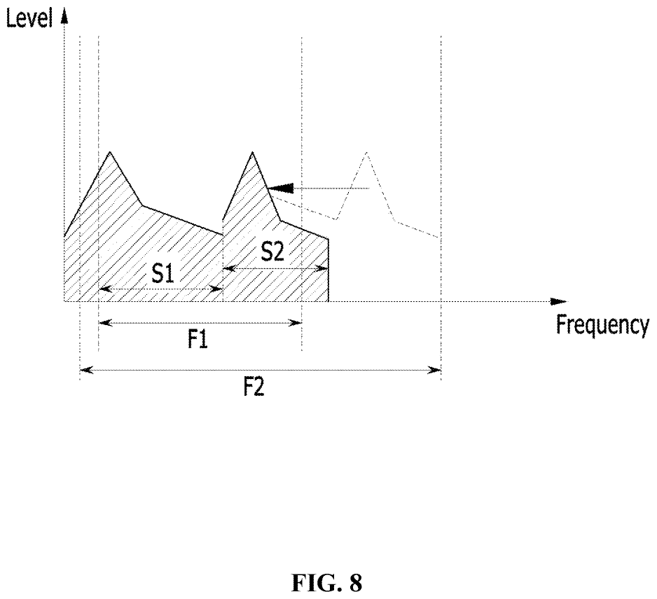

FIG. 8 is a diagram illustrating another method of modulating the sound signal in the method of controlling the cochlear implant system according to an embodiment of the present disclosure.

Referring to FIG. 8, the method of controlling the cochlear implant system according to an embodiment of the present disclosure may modulate the user-unrecognizable signal S2, which the user cannot recognize among the sound signals of the audible frequency F2, into the range of the user-recognizable frequency F1.

For example, in a state in which the user-recognizable signal S1 included in the range of the user-recognizable frequency F1 is maintained as it is, the frequency of the user-unrecognizable signal S2 may be changed into the range of the user-recognizable frequency F1.

In this case, in the process of changing the frequency of the user-unrecognizable signal S2 into the range of the user-recognizable frequency F1, the frequency of the user-recognizable signal S1 may be changed so as not to overlap the user-recognizable signal S1.

For example, by reducing the user-recognizable signal S1 included in the range of the user-recognizable frequency F1 at a predetermined ratio, an available frequency band in which the user-recognizable signal S1 is not present may be generated, and the frequency of the user-unrecognizable signal S2 may be changed into the available frequency band. Accordingly, the user may listen to sound having clearer quality.

The transmitting of the modulated signal to the internal device 100 (S500) is a process of transmitting the sound signal modulated into the range of the user-recognizable frequency F1 to the internal device 100 through the second coil 210.

Accordingly, the internal device 100 delivers the electrical signal received through the first coil 130 to the active electrode 110, thereby allowing the user to hear through stimulation of the cochlea.

The detecting of the state information of the internal device 100 using the second test signal (S600) is a process of detecting a defective state of the internal device 100 implanted in the body of the user.

The sound processor 200 may transmit the second test signal to the internal device 100 at predetermined periods, and may detect a mutual induction signal generated in the second coil 210 thereby to detect the state information of the internal device 100.

For example, when inflammation or an ulcer occurs around the active electrode 110 or the reference electrode 120 of the internal device 100, a level of the mutual induction signal generated in the second coil 210 by the second test signal may significantly change.

Accordingly, the sound processor 200 may determine the state of the internal device 100 according to the level of the mutual induction signal.

The generating of the notification signal according to the state information of the internal device 100 (S700) is a process of determining that a defect has occurred in the internal device 100 and generating the notification signal when the mutual induction signal caused by the second test signal is changed to a reference level or less.

As a result, the cochlear implant system of the present disclosure determines whether the internal device 100 implanted in the user is defective and generates the notification signal to the user, thereby allowing the user to easily determine a time for replacing or repairing the internal device 100.

The above description and the accompanying drawings merely illustrate the technical spirit of the present disclosure by way of example. A person having ordinary skill in the art to which the present disclosure pertains may make various modifications and variations, such as combination, separation, substitution, and change of configurations, without departing from the essential features of the present disclosure. Therefore, the embodiments disclosed in the present disclosure are not intended to limit the technical spirit of the present disclosure, but are intended to describe the technical spirit of the present disclosure, and the scope of the technical spirit of the present disclosure is not limited by these embodiments. That is, within the scope of the present disclosure, all components thereof may selectively operate in combination with one or more components. The scope of protection of the present disclosure should be interpreted by the following claims, and all technical spirits within an equivalent range thereof should be interpreted as being included in the scope of the present disclosure.

### DESCRIPTION OF REFERENCE NUMERALS

100: internal device
110: active electrode
120: reference electrode
130: first coil
200: sound processor
210: second coil
220: microphone
230: sound modulator
240: controller
250: transmitter
260: characteristic detector

What is claimed is:

1. A cochlear implant system comprising:
an internal device configured to be implanted in an outer ear of a user, the internal device including a first coil; and
a sound processor configured to modulate a sound signal according to hearing characteristics of the user and transmit the modulated sound signal to the internal device in a non-contact manner.

2. The system of claim 1, wherein the sound processor comprises:
a second coil configured to exchange electrical signals with the first coil through mutual induction;
a microphone configured to receive the sound signal;
a sound modulator configured to divide the sound signal into a plurality of frequency bands and modulate signals in the plurality of frequency bands according to the hearing characteristics of the user;
a transmitter configured to transmit the modulated signals in the plurality of frequency bands to the internal device through the second coil;
a characteristic detector configured to detect an induced signal induced in the second coil by a test signal and determine the hearing characteristics of the user or a state of the internal device based on the induced signal; and
a controller configured to control the sound modulator, the transmitter, and the characteristic detector.

3. The system of claim 2, wherein the test signal comprises:
a first test signal for detecting the hearing characteristics of the user; and
a second test signal for detecting whether the internal device is defective.

4. The system of claim 3, wherein the first test signal is provided at an initial operation time after the internal device is implanted.

5. The system of claim 3, wherein the second test signal is provided at predetermined periodic intervals.

6. The system of claim 4, wherein the sound modulator is configured to, based on the hearing characteristics of the user detected using the first test signal, divide the sound signal into:
a user-recognizable signal included in a user-recognizable frequency range; and
a user-unrecognizable signal included in a user-unrecognizable frequency range outside the user-recognizable frequency range.

7. The system of claim 6, wherein the sound modulator is configured to modulate the user-unrecognizable signal into the user-recognizable frequency range.

8. The system of claim 7, wherein the sound modulator is configured to modulate the user-unrecognizable signal such that the user-unrecognizable signal overlaps the user-recognizable signal.

9. The system of claim 7, wherein the sound modulator is configured to reduce the user-recognizable signal at a predetermined ratio and modulate the user-unrecognizable signal into an available frequency band in which the reduced user-recognizable signal is not present.

10. A method of controlling a cochlear implant system including a sound processor configured to communicate with, in a non-contact manner, an internal device implanted in an outer ear of a user, the method comprising:
transmitting a first test signal to the internal device to detect hearing characteristics of the user;
determining a modulation algorithm for a sound signal according to the hearing characteristics of the user;
receiving the sound signal through a microphone;
modulating the sound signal according to the modulation algorithm; and
transmitting the modulated sound signal to the internal device.

11. The method of claim 10, further comprising:
transmitting a second test signal to the internal device to detect state information of the internal device; and
generating a notification signal according to the state information of the internal device.

12. The method of claim 10, wherein modulating the sound signal comprises, based on the hearing characteristics of the user detected using the first test signal, dividing the sound signal into:
a user-recognizable signal included in a user-recognizable frequency range; and
a user-unrecognizable signal included in a user-unrecognizable frequency range outside the user-recognizable frequency range.

13. The method of claim 12, wherein modulating the sound signal comprises modulating the user-unrecognizable signal into the user-recognizable frequency range.

14. The method of claim 13, wherein modulating the sound signal comprises modulating the user-unrecognizable signal such that the user-unrecognizable signal overlaps the user-recognizable signal.

15. The method of claim 13, wherein modulating the sound signal comprises:
reducing the user-recognizable signal at a predetermined ratio; and
modulating the user-unrecognizable signal into an available frequency band in which the reduced user-recognizable signal is not present.
